(19) United States
(12) Patent Application Publication
(10) Pub. No.: US 2009/0221896 A1
(43) Pub. Date: Sep. 3, 2009

(54) Probe For Data Transmission Between A Brain And A Data Processing Device

(76) Inventors: Jörn Rickert, Freiburg (DE); Carsten Mehring, Freiburg (DE); Tonio Ball, Freiburg (DE); Ad Aertsen, Freiburg (DE); Andreas Schulze-Bonhage, Freiburg (DE)

Correspondence Address: IP STRATEGIES, 12 1/2 WALL STREET, SUITE E, ASHEVILLE, NC, 28801, US

(21) Appl. No.: 12/280,415

(22) PCT Filed: Feb. 12, 2007
(86) PCT No.: PCT/EP2007/051359

§ 371 Date: Dec. 31, 2008

(30) Foreign Application Priority Data
Feb. 23, 2006 (DE) 10 2006 008 501.9

(51) Int. Cl. A61B 5/0478

(52) U.S. Cl. 600/378; 600/544

(57) ABSTRACT

The invention relates to a probe for data transmission between a brain and a data processing device. Said probe has a support with electrodes fitted thereto. Said electrodes can be made to electromagnetically interact with neurons of the brain for the purpose of detecting neuronal activity and/or the transmission of stimuli and can be coupled to the data processing device. The shape of the support can be adapted to an inner surface of the brain to such a degree that it can be inserted into the interior of a sulcus of the brain.

The invention relates to a sensor for data communication between a brain and a data processing device, as well as to a method of producing such a sensor. In addition the invention relates to a means comprising such a sensor, respectively to a method for data communication between a brain of a living being and a data processing device.

In sensing neuronal activity in the brain of a living animal or human being there is the problem of it being very difficult to obtain activity patterns with good resolution in time and space without tissue injury. Techniques in which the sensing electrodes can remain outside of the skull on the surface of the head (electroencepthalography, EEG) are restricted to sensing the activity of larger neuron populations with relatively poor three-dimensional detail.

Electrodes implanted in brain tissue in thus gaining direct access to the detecting neurons furnish the most precise activity data, but at the cost of injuring nerve tissue and destroying nerve connections. Due to the neurons being intermeshed to a high degree this always involves the risk of important neuron functions being restricted. Although this injury may still be acceptable in scientific work on animals, when a therapeutical application on the human brain is involved it is then at the latest that there is the dilemma of having to weigh the pros and cons of how useful the therapy is and how injurious the electrodes are.

One partial solution is to use, instead of electrodes implanted in nerve tissue, electrodes which although require an opening in the skull are merely located on the surface of the brain with no injury to the actual brain tissue. In this way the sensed signals are dominated by the neuron areals in the direct vicinity of the outer surface of the brain.

The drawback of this partial solution is as already mentioned: regions of the brain not directly located at the outer surfaces remain only partly attainable. Thus, although this is an improvement over conventional techniques sensing outside of the skull, sensing still remains restricted.

In addition to detecting neuronal activity by sensing the electrical potential resulting from the activity of the neurons, the converse approach can also be of interest, i.e. stimulating neurons with electrical pulses. However, the crux of the problem as explained above remains: the stimulation electrode, just like the detection electrode, needs to gain access to the corresponding neurons for their specific stimulation.

One area of application in which precise mapping neuronal activity plays a major role involves the more recent advances in motoric neuroprosthetics aimed at paralyzed patients for whom an organic healing is no longer possible, although the cortex or at least the motor cortex as the areal substantial to controlling intentional actions is at least partly still intact, but the nerve endings to the muscular system are disrupted—or the muscular system/limbs no longer exist. The salient and most frequent cause of such paralysis are ischemical infarcts of the brain or intracerebral hemmoraghes (“stroke”).

Particularly serious is the case of locked-in patients robbed of any intentional possibility of action due to a total paralysis of the sceletal muscular system (for example due to aymotrophic lateral sclerosis (ALS)/muscular degradation) or a stroke in the region of the cortex. Such patients, although fully conscious of what is wrong with them cannot do anything about it. This is just the same with patients having lost an extremity or are paralyzed from the neck down, here too they being prevented from implementing intended actions.

Motoric neuroprosthetics are designed to attain, reattain or improve activities by the intentional activation of a prosthetic by means of native brain signals. The basic requirement for this is precisely mapping neuronal activity, in this case in the motor cortex. But, the reverse case is likewise involved in which sensorial signals of paralyzed parts of the body, such as a touch, for instance, fail to reach the brain. Here, stimulating the neurons of the responsible areal of the brain—for example the somatosensorial cortex—can replace the body's own disrupted signal transfer.

In all of the cases as described the conventional techniques are hampered either by the lack of precision of prediction or by they injuring intact brain tissue to such a degree that in animal experimentation the results are detrimented and on humans therapy is restricted or even prevented.

The object of the invention is thus in avoiding injury of brain tissue to achieve precise access to a large neuron population.

This object is achieved by a sensor as set forth in claim 1 and respectively by use thereof in a device as set forth in claim 9 or a method as set forth in claim 23. A method of producing a sensor in accordance with the invention is claimed in claim 16. The achievement in accordance with the invention is based on the principle of exploiting the morphology of the brain and adapting the sensing/stimulation electrode instead of the inversion by an invasive operation to subject the brain tissue to the shape of the electrode with resulting injury by sensing/stimulation.

In accordance with the invention there is thus provided a sensor for data communication between a brain 2 and a data processor 5, the sensor comprising a substrate 1a to which electrodes 1c, 1d are applied for sensing neuronal activity and/or the transfer of stimulation in electromagnetic interaction with neurons of the brain 2 and which can be coupled to the data processor 5, the substrate 1a being shapeable to conform with an inner surface of the brain 2 such that it can be implanted into an interior of a sulcus 2c of the brain 2 wherein the substrate 1a is configured flexible and comprises two surfaces facing each other, on at least one the surfaces at least one array of electrodes 1c, 1d is applied, the electrodes 1c, 1d being configured as contact pads such that the at least one array of electrodes 1c can electromagnetically interact with neurons of at least one sidewall 2a of the sulcus 2c, the electrodes 1c, 1d being adaptable in their array to the morphology of the at least one sidewall 2a.

The electrodes are thus configured sheeted or punctiform enabling the sensor to attain the neurons of both sidewalls in stimulating or detecting them depending on the activation. This now makes it possible for the sensor to attain a particularly large population of neurons located on both sides of the sulcus as would be totally impossible with a surface electrode and with an invasive electrode only with complications with corresponding brain tissue injury.

This achievement has the advantage that the electrodes and the sensor leave the brain uninjured in thus also diminishing the diverse risks involved in an operation. At the same time, the long-term compatibility is good, there being hardly any risk of the electrodes being jolted out of place because the substrate is shaped to conform with the sulcus 2c and thus adapted to the individual fissures and windings of the brain for a snug, secure fit. This results in the signals remaining stable because the adjoining neurons are always the same, and also with the complete absence of sharp edges or tips which could cause injury should the substrate become displaced, for instance due to sudden movements in an accident. These injuries may occur even with normal movements where the electrodes have been conventionally implanted intracortically. In conclusion, this achievement in accordance with the invention now makes it possible, however, to gain access to areals of the brain and particularly of the cortex as are of interest or even a necessity for the applications as described below.

To advantage the neurons in the first and second sidewall belong to different function areals of the brain, the sulcus in this case dividing two function areals, enabling a separate function areal to be activated from each side of the sensor.

In one advantageous further embodiment the neurons of the first sidewall belong to the motor cortex and the neurons of the second sidewall belong to the somatosensorial cortex. The motor cortex is a typical output-oriented areal, conversely the somatosensorial cortex is an input-oriented areal. One and the same substrate in this further embodiment can serve both motoric detection and somatosensorial stimulation.

Preferably the first array comprises detection electrodes and the second array stimulation electrodes. This assignment is particularly of advantage when detection electrodes are assigned to an output-oriented areal and stimulation electrodes to an input oriented areal. But in spite of this, this divisioning must not be exclusive, because simulating an output-oriented areal or detection from an output-oriented areal may be appropriate.

Preferably the substrate is made of polyimide or silicone, these materials having a proven record of success in being condusive to processing, biocompatible and feature a long-term stability.

Preferably a plurality of electrodes having a density between one and 1,000 electrode contacts per cm² are applied to the substrate, although, of course, this upper limit of 1,000 electrode contacts per cm² can be elevated, as long as the corresponding technology is selected and as required by the application. Depending on the neuron population of interest the balance between three-dimensional resolution, on the one hand, and cortex cerebri as well as the electrode sensitivity, on the other, can be selected.

Preferably electrodes 1c, 1d are made of gold, platinum, a metallic alloy, conductive plastics or semiconductor materials, it being particularly the metals that are well suited because of their good sensing/stimulation results and their long-term stability and comparability, whereas conductive plastics or semiconductor materials can be processed particularly well with the flexible substrate.

The means for data communication comprising at least one sensor in accordance with the invention is configured to advantage to activate a first part of the electrodes by reading out the input signals of the detection electrodes and a second part of the electrodes by means of feeding output signals as stimulation electrodes so that a two-way exchange of data is made possible, in thus exploiting the possibility of the sensor to activate the electrodes in one of both directions. Each electrode can sense both neuronal activity as well as electric pulses, one of these roles being assignable as required to the electrodes when activated in this way. The means thus permits not just one way of transfer but both. Conventionally, this would have necessitated such a large number of invasive electrodes that the overall gain becomes doubtful. Just a single surface electrode attaining various areals for stimulation and detection is likewise difficult to imagine, it needing to be at least split in two to avoid it becoming oversized which, of course, poses problems in the operation, positioning and as to long-term stability.

Preferably the analyzer is additionally configured as an effector controller of a connectable effector and computes on the basis of the input signals effector control signals for the effector and/or computes on the basis of the effector condition signals of the effector the stimulation signals. In other words, the electromagnetic signals from the neurons are not just mapped but can be made use of directly for controlling an effector. Conversely, the effector can tweak the neuronal activity in this way.

Preferably the means is configured such that
the effector is a prosthetic;
the input signals are those of neurons in the motor cortex and the stimulation signals are for neurons in the somatosensorial cortex;
the effector control signals tweak activation parameters of the prosthetic and
the effector condition signals are position, action and/or condition parameters of further sensors such as pressure, tactile, spacing or temperature sensors
   so that the brain can control activation of the prosthetic and directly receives somatosensorial feedback as to the action and ambience of the prosthetic

In this way the patient has the possibility of not just intentionally controlling the prosthetic, he also receives a sensorial feedback, i.e. a feeling for the body part he employs.

As an alternative the means is configured such that
the effector is a body part of the living being,
the input signals and those of neurons in the motor cortex and the stimulation signals are for neurons in the somatosensorial cortex;
the effector control signals tweak motor neurons or muscle fibers of the body part and
the effector condition signals are signals from receptors or receptor neurons of the body part and/or position, action and/or condition parameters of further sensors such as pressure, tactile, spacing or temperature sensors
   so that the brain can control the action of the body part and directly receives somatosensorial feedback as to the action and ambience of the body part.

In this way the control of the body part both as regards its activity and as regards the feeling thereof are returned.

Again as an alternative the means is configured such that
the effector is a computer particularly including a display;
the input signals and those of neurons in the motor cortex and the stimulation signals are for neurons in the somatosensorial cortex;
the effector control signals tweak virtual, particularly displayed actions or functions in the computer and
the analyzer computes on the basis of the virtual action or function the effector condition signals.

The virtual effector has the major advantage of being extremely variable in its functionality in thus being made available at low cost and practically with no limits in being freely configurable and with total freedom from mechanical problems of any kind. Even if the detected signals are lacking in quality a very useful function can still be achieved in this case and feedback thereof made available.

To advantage an amplifier is provided configured for amplifying and filtering input signals into preprocessed input signals and/or output signals into stimulation signals. The neuron signals detected by the electrodes often require conditioning before their analysis can be commenced. Conversely the stimulation signals must, of course, be of a quality as can be processed for the neuron.

In the method in accordance with the invention for producing a sensor to advantage the geometry of the sulcus is mapped by analysis of non-invasive imaging techniques including computer tomography (CT) and magnetic resonance tomography (MRT) as well as functional magnetic resonance tomography (fMRT) and similat techniques as known from research and development. The advantage here is that, on the one hand, the patient is spared a further operation before later insertion of the sensor and, on the other, avoiding the operation being drawn out or the quality of the sensor reduced because of shaping needs to be done under time pressure during the operation. But apart from this, excellent conformity is made possible, of course, by the geometry being so well known from imaging.

Preferably the electrodes are arranged on the substrate in a way as adapted to the morphological of the interior in thus adapting not just the substrate itself but also the actual information carriers to the cerebral requirements involving both the geometry as such as well as other morphological demands, for instance neuron density or size and their degree of interlinking, the strength of their electromagnetic fields or the like as can then be simulated in the arrangement, size, sensitivity etc. of the electrodes.

Further features and advantages similar to those of the sensor itself as described above, but not conclusive, read from the sub-claims following that of the method of production.

Also, the method of data communication in accordance with the invention comprising a sensor in accordance with the invention inserted in a sulcus shows similar and further features and advantages as described by way of example, but not conclusively in the subsequent sub-claims.

The invention will now be detained also as regards further features and advantages with reference to the attached drawing in which:

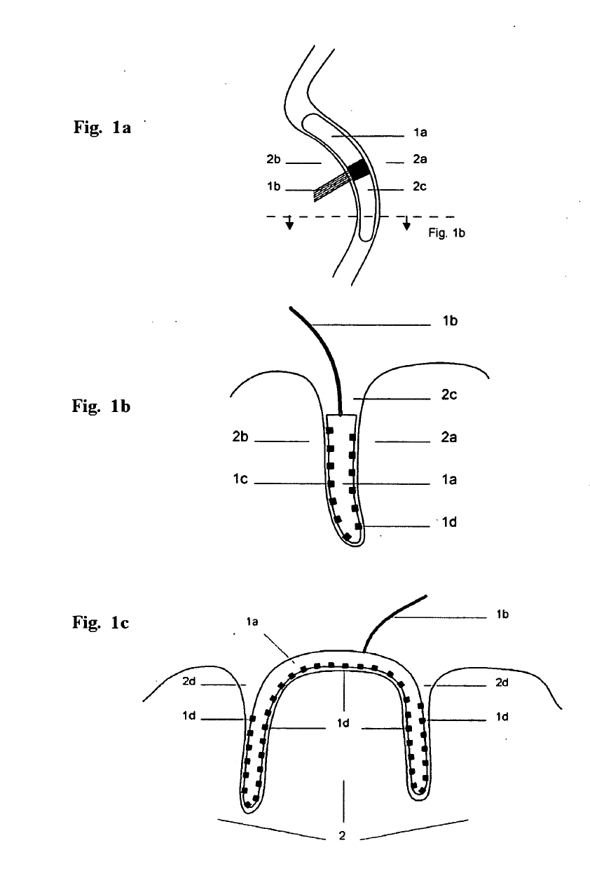

FIG. 1a is a top-down view of one embodiment of the invention implanted in the brain;

FIG. 1b is a cross-sectional view of the embodiment as shown in FIG. 1a as taken along the broken line in FIG. 1a;

FIG. 1c is a cross-sectional view of an alternative embodiment of the invention;

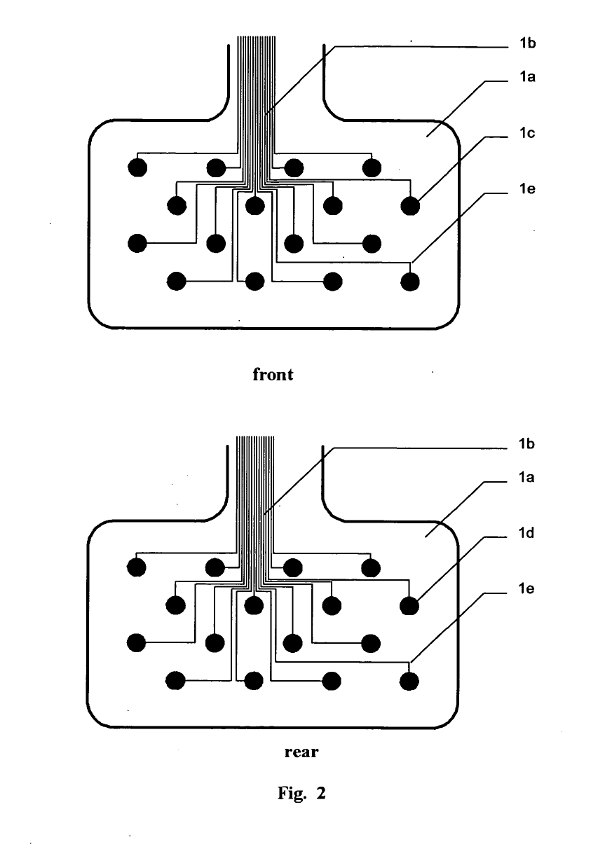

FIG. 2a is a side view of the front substrate surface and electrodes of the embodiment as shown in FIG. 1;

FIG. 2b is a side view of the rear substrate surface and electrodes of the embodiment as shown in FIG. 1;

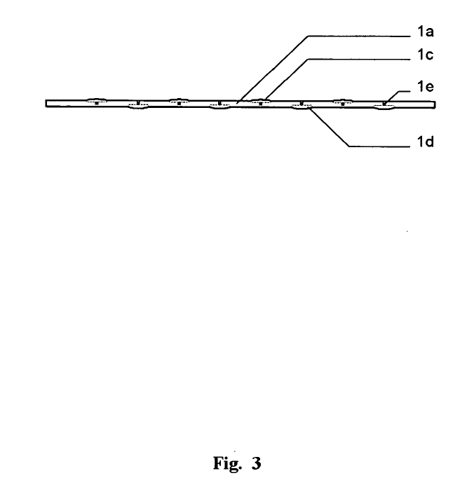

FIG. 3 is a section view of the substrate as shown in FIG. 2;

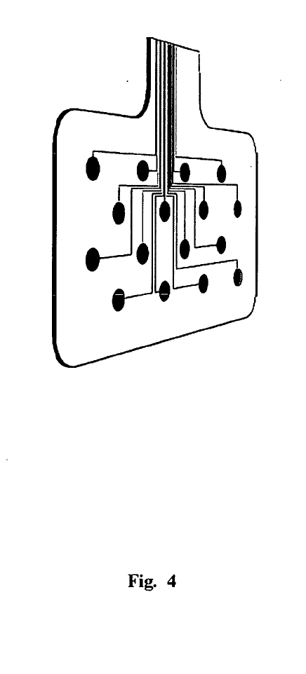

FIG. 4 is a view in perspective of the substrate;

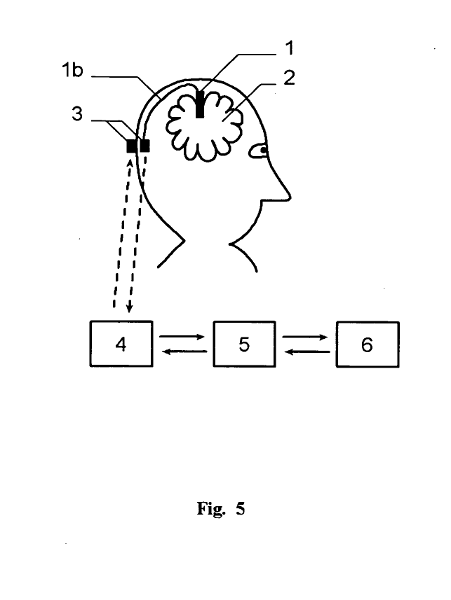

FIG. 5 is an overview of on implanted embodiment of the invention and advantageous periphery;

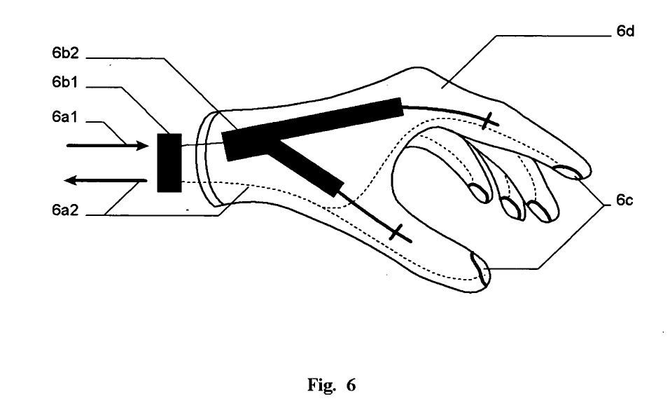

FIG. 6 is an illustration of an arm prosthetic as an example of an effector as can be activated by one embodiment of the invention;

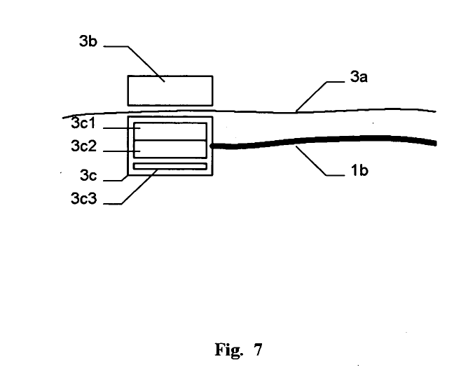

FIG. 7 is a diagrammatic view of the stimulation for one embodiment of the invention;

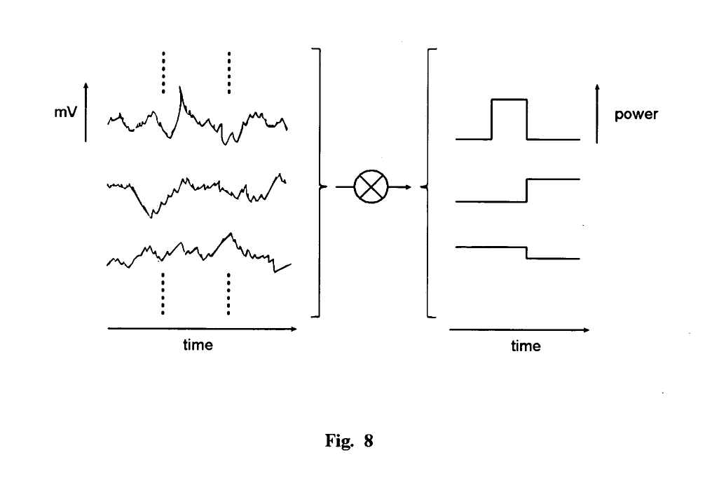

FIG. 8 is a diagrammatic view as an example for the conversion of neuron signals into control signals for an effector;

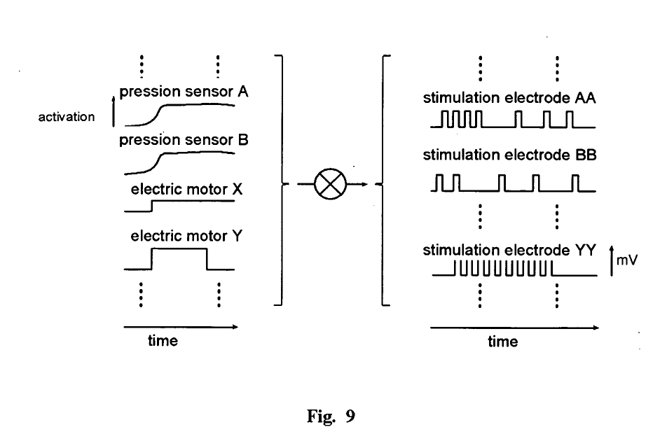

FIG. 9 is a diagrammatic view as an example for the conversion of feedback data of an effector into stimulation signals for the electrodes; and

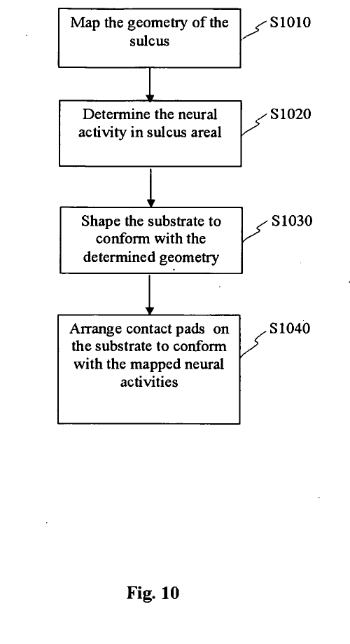

FIG. 10 is a flow diagram of the method of production in accordance with the invention.

The cortex cerebri of the human brain is highly convoluted in shape in which sulci (fissures) separate the gyri (convolutions) from each other. It is emphasized that although the medical applications are primarily focussed on the human brain, the invention is not restricted to this application but is pertinent to any gyrencephalic animal brain (i.e. having fissures and convolutions) and not necessarily exclusively for therapeutic purposes but also for neuro-scientific purposes.

Referring now to FIGS. 1a to 1c there is illustrated an implanted embodiment of the invention showing how it is sited in the brain in a top-down view and cross-sectional view. Embedded in a sulcus 2c defined by two side surfaces of the adjoining convolutions 2a, 2b is a multi-electrode 1 comprising a substrate 1a of a flexible or elastic material. The multi-electrode 1 is accordingly a corticomorphous electrode adapted, or self-adapting, to the shape of the surface of the brain.

This is why the substrate 1a is shaped to precisely conform with the surface shape of the convolutions of the brain for a snug fit. For a stable site it is good practice to implant the substrate 1a down to the bottom of the sulcus 2c, but this is not a mandatory requirement if higher level side surfaces are to be contacted for which the height of the substrate 1a is insufficient. Polyimide or silicone are suitable materials for the substrate 1a because of their comparability whilst being easy to work and their insensitivity, although any other material is just as suitable, as long as it has the necessary flexibility and biocompatibility. And, of course, the material needs to be conductive, but in any case it must be easy to shape the substrate to individual requirements, for example, by being cut to size. In conclusion, the substrate 1a must be elastic and sufficiently thin, mostly with a thickness <<1 cm with rounded edges so as not to injure tissue.

Applied to the substrate 1a is an array of electrodes 1c, 1d, each of which is connected by a lead 1b for conducting signals to the ambience. The precise structure of the electrodes 1c, 1d on the substrate 1a and how they are wired is detailed below. Due to the snug configuration of the substrate 1a the electrodes 1c, 1d surface applied thereto come into contact directly with surface of the brain 2a, 2b to thus have excellent electromagnetic interaction with the neurons of the adjoining brain tissue 2a, 2b. Although the brain tissue 2a, 2b is stimulated in the one signal direction by electrode contacts or their activity sensed in the other direction via the electrical potential, this must not be taken to mean that the invention is restricted thereto. Thus, the invention also covers stimulating by potential, sensing or tweaking currents or any other electrical or magnetic parameter, it merely being important that each electrodes 1c, 1d can sense or stimulate the activity of the neurons by means of electromagnetic pulses depending on how activated.

One special embodiment of the sensor is devised for the central sulcus 2c between the primary somatosensorial cortex 2a and primary motor cortex 2b. In this case the roles of the electrodes 1c, 1d are assigned so that the electrodes 1c in contact with the surface of the primary motor cortex 2b are activated as sensing or detecting electrodes 1c whilst the electrodes 1d in contact with the surface of the primary somatosensorial cortex 2a are activated as stimulation electrodes 1d. Although this assignment is in keeping with the task of the somatosensorial cortex 2a which in an intact brain mainly processes incoming information whilst the primary motor cortex 2b is responsible for planning and implementing activation and thus, functionally, communicates output signals to the adjoining parts of the brain and backbone, it is just as possible to allocate the electrodes 1c, 1d differently in, for instance, providing for stimulation in the primary motor cortex 2b. Just as feasible would be e.g. to trigger stimulate, test, support or intensify a functional neuron activity pattern in the primary motor cortex 2b. In other words, this is a question of the how activated and applied so that the invention is not restricted to a rigid allocation as described.

Although particular attention is given to application at the somatosensorial cortex 2a and primary motor cortex 2b as an important example thereof, the invention is not restricted to this, the sensor in accordance with the invention being basically suitable for any sulcus and curved electrodes can also be adapted to any convolution of the brain and thus extend from one sulcus into an adjoining sulcus. This case is illustrated diagrammatically in FIG. 1b showing the substrate implanted in a sole sulcus, a cross-section of which is shown analogously in FIG. 1c.

An operation with which the substrate 1a is implanted requires a specific presurgical diagnosis and planning, one of the salient aspects of which is to define the precise site for the implant which because of the strong inter-individual neuroanatomical variability of the human brain cannot be defined a priori. Only in exceptional cases would siting an implant be wanted which was not individually defined beforehand. Although from general mappings of the brain it is known where functional areals are to be found, indeed even the human anatomy is mapped and individual parts of the body are assigned to spatially distinct regions of the cortex in the special example of the motor cortex and the somatosensorial cortex, this prior information usually lacks sufficient precision for the individual patient.

This is why pin-pointing sites before the operation is done individualized for the patient by fMRT in which the activation of the brain specific to the site concerned is sensed whilst the patient attempts, imagines or observes control of the effector in thus enabling the implantation site to be defined three-dimensionally highly accurately. This can be followed to further enhance siting by an EEG with subsequent source reconstruction whilst the same motor paradigmens (attempting, imagining or observing effector control) are performed.

By way of anatomical MRT imaging the three-dimensional geometry of the sulcus 2c is mapped, with the aid of which the substrate 1a is shaped to precisely conform to the gap or interior of the sulcus 2c in rendering the implant impervious to movements of the head in keeping it in good contact with the sidewalls 2a, 2b, whereby a certain error tolerance exists by the flexibility of the brain tissue.

It is, of course, just as possible to apply the substrate 1a without these complicated preprocedures, though seldom even in animal experimentation, this is, of course, less than optimum for patients. But the invention is not at all intended to exclude this, solely adapting the shape of the substrate 1a being the one mandatory requirement. This, however, must not necessarily be based on mapping the brain of the individual concerned, but e.g. it may be based on what is expected, predicted in theory or known from experience.

Referring now to FIGS. 2a and 2b the configuration of the substrate 1a and the arrangement and connections of the electrodes 1c, 1d will now be explained, FIG. 2a showing the front, FIG. 2b the rear side of the substrate 1a. These FIGs. relate to the example of an embodiment in which a surface of the substrate 1a is in contact with an areal of the brain to be stimulated and the other with an areal of the brain to be sensed, it being, however, understood that the invention is not restricted to this but is compatible with any other arrangement of the electrodes 1c, 1d and their connections.

The substrate 1a is depicted roughly rectangular in shape as may be sufficient in application and it may be devised, for example, as a single or double film. But in an embodiment adapted to the sulcus 2c the material of the substrate is modelled so that the configuration conforms with the boundaries of the sulcus 2c, it needing to be noted that the sulcus 2c permits application of the substrate 1a only when extremely thin.

Usually, the substrate 1a is made of a flexible material. If the substrate 1a is correspondingly premodelled, other materials come into consideration as long as they do not make it a problem inserted it into the sulcus 2c. But in any case the material needs to be biocompatible, i.e. non-detrimental to the brain tissue even in a long-term use. Although polyimide or silicone is a suitable substrate material for this purpose it is understood that the invention is not restricted to this material.

The electrodes 1c, 1d as contact points or pads take the form of a matrix. The surface of the substrate 1a with the contact points or pads is configured substantially flat. Conductors 1e in the interior of the substrate 1a connect each electrode 1c, 1d individually and without overlapping their individual conductors 1e to the lead 1b for signal exchange. The lead 1b is devised at least two-part, one sensing lead 1b1 conducting signals of the electrodes 1c to the exterior and a stimulation lead 1b2 conducting signals for the stimulation electrodes. However, it is just as possible to use a one-part lead 1b for communicating sensing data to the exterior and stimulation data to the interior in differing time intervals. The person skilled in the art is aware of how these conductors 1e are made and how they can be arranged.

The electrodes too can be made of various materials, particularly gold, platinum or metallic alloy or also of conductive plastics as well as semiconductor materials. The substrate 1a may be one to more than ten centimeters large. The electrode contacts are designed for a typical density of 1 to more than 10,000 electrode contacts per cm². The higher the density of the electrode contacts the better the signal resolution, but, of course, this adds to the complications not only in making the electrode electrodes 1c, 1d but also in amplification and the computational complexities in controlling activation.

It is, of course, just as possible that the arrangement differs from that as shown with opposing rows, practically any arrangement of a dot array on a surface area being possible.

As evident from e.g. FIGS. 1b and 1c the two arrays of electrodes must not necessarily be arranged symmetrical to a section plane through the substrate 1a. Instead, the electrodes of the one surface can be arranged staggered relative to the electrodes of the other surface or any other arrangements in accordance with the results of fMRT analysis, it also being just as possible that one surface of the substrate 1c is totally or partly void of electrodes, as illustrated e.g. in FIG. 1c.

To particular advantage this substrate, unlike implanted electrodes, does not injure brain tissue, this also achieving a better long-term stability in sensing the signals because electrodes penetrating brain tissue result in localized destruction of tissue and thus possibly in ruining local neuronal activity. Depending on the particular applications the substrate 1a with the electrode electrodes 1c may also be very small (<<1 cm). In this case the operation by which the substrate 1a is implanted in the patient has fewer complications with far less injury to the patient.

Referring now to FIG. 3 there is illustrated a section view through the substrate, i.e. in profile, making it evident how the substrate has two flat surfaces.

Referring now to FIG. 4 there is illustrated the substrate 1a in a view in perspective.

The sensors are engineered as described in the following, i.e. individualized to conform with the brain of the patient.

Mapping the exact anatomy of the cortex cerebri of the brain is done by structural imaging techniques, preference being given to TI weighted MRT images since these can be obtained without exposing the patient to ionizing radiation. These techniques also map areals of the brain controlling intentional activities, especially those of functional MR imaging (fMRT) being of advantage because of their excellent three-dimensional resolution.

The information provided by structural and/or functional imaging techniques can be put to use to adapt the following properties of the electrodes to be implanted, sited precisely in the brain of the patient receiving individual treatment, cf. also FIG. 10:
size of the sensor
shape of the sensor
arrangement of the individual contact pads on the substrate 1a of the sensor
number of sensors to be implanted in all
positioning the sensor on the cortex.

Forming the basis for this is a highly resolved structure set of image data of the brain, preferably with a resolution of 1 mm×1 mm×1 mm or better. In addition, functional image data are mapped during a test battery of activation tasks capable to covering the full repertoire of natural activation tasks ultimately to be controlled by the BMI.

In the production method in accordance with the invention the following steps are performed:

Mapping the geometry of the sulcus 2c from analysis of the set of structural image data (step 1010)
Utilizing the functional image data to determine the neuronal activities of the sulcus (step 1020) particularly by some or all of the following steps: correcting the effects of movements of the head during sensing, eliminating artifacts, standardizing in a system of standard coordinates, three-dimensional filtering, temporal filtering, statistical analysis on the basis of parametric or also non-parametric techniques.

Then, from the resulting activation data, during various activation tasks and—optionally—additionally taking into account the structural data, the areal(s) of the brain is/are determined which have the highest anticipation of activation information. By corresponding algorithms an optimum implantation is designed achieving maximum activation information for a minimum of sensors to be implanted or connecting a minimum total surface in the sensors to be implanted. In this step, all of the parameters as recited above can be involved. The data as to the parameters of the individual substrates are then used—in step 1030—for individual production of the susbstrates to be implanted.

In step 1040 the contact pads are then positioned on the substrate 1a such that they correspond to the sites of significant neuronal activity in the areal of the sulcus where the substrate 1a is to be sited in keeping with the results of steps 1010 and 1020.

In other words, the method of production furnishes a sensor having a substrate 1a featuring a specific geometry in shape and a specific arrangement of contact points/pads.

Where necessary for neurosurical aspects the data of the implantation optimized in the previous step is communicated to the neuronavigational device and siting the sensor in the brain performed computer-assisted.

Referring now to FIG. 5 there is illustrated an overview of one embodiment of the invention as used in the brain 2 with an advantageous periphery showing how the multi-electrode 1 for detecting the neuronal activity or stimulation is implanted in the skull of the patient as described above in a sulcus 2c. The multi-electrode 1 senses the neuronal activity and communicates it via a signal interface 3 (described below) as electromagnetic input signals to an amplifier 4 preferably configured as a multichannel amplifier, involving in addition to amplification, high, low or bandpass filters (for example Savitzky-Golay, Butterworth or Chebychev filters). Of advantage is a high temporal resolution for real time communication, ideally with a sampling rate of better than 200 Hz, although lower values are not excluded.

The amplifier amplifies and filters the electromagnetic input signals and passes on the thus preprocessed signals in real time to an analyzer chip, a computer or like system 5 to the signal processor. In one embodiment of the invention this already achieves the one aim of having sensed the neuronal activity for analyzing in the system 5 as desired.

In another embodiment of the invention stimulation signals are generated in the system which are supplied via the amplifier 4 and the signal interface 3 to the multi-electrode 1, the individual electrodes 1d of which output the corresponding stimulation pulses.

In yet another embodiment the system 5 communicates the effector control signals signals to an effector 6; conversely the effector 6 can return effector condition signals to the system 5.

It is important to note that further combinations of the cited components are just as possible, for instance connecting the effector controller may be two-way, although the effector can also be prompted to act in one way exclusively for actions or communicate exclusively condition signals (as a straight sensor). It is just as possible to engineer the connection between the system 5 and the multi-electrode 1 two-way, depending on the application, or one-way in one of the two directions. Preferred, however, is the two-way connection since then the inventive arrangement of the multi-electrode 1 can be best exploited within a sulcus 2c.

Representative for the wealth of possible application variants in which the multi-electrode 1 is implanted in differing areals of the brain, the following describes implantation in the central sulcus 2c between the somatosensorial cortex 2a and the primary motor cortex 2b. This is not at all to be appreciated as being restricted thereto, the invention also encompassing the possibility of stimulating and/or detecting any other areal of the brain.

The effector 6 may be any of the three groups as cited above, i.e. a mechanical device such as a robotic appliance, robotic arm or a prosthetic, a native part of the body or an electrical device activated by virtual command of a computer such as a computer, a mobile communications device, a household appliance or the like.

Referring now to FIG. 6 there is illustrated diagrammatically a hand prosthetic to assist in explaining the first case of a prosthetic, in other words an artificial limb but, of course, it will be appreciated that any kind of prosthetic can be activated, feasible being even such absurd activations as for a third arm or leg.

Via an effector input lead 6a1 the signals for controlling the effector are communicated by the system 5 to the effector 6.

The prosthetic comprises a rotation system 6b1 for turning the hand. A controller for a motor of the rotation system 6b1 turns the prosthetic in accordance with the effector control signals. In addition, the prosthetic comprises a gripper system 6b2 including a motor and controller which performs the opening and closing actions of a finger part of the hand in accordance with the effector control signals. It is to be noted that no attempt has been made to inform all details of the controller from the neuronal data; instead the system 5 could also just predict the nature of the intended action to then automatically determine the single steps as needed.

For generating a functional feedback to the brain, pressure sensors 6c are attached to the finger part, the signals of which indicating the condition of the effector are fed back via the effector output lead 6a2 to the system 5. In conclusion the prosthetic is enclosed by a cladding expediently having the appearance of a human hand. It is to be noted that a hand prosthetic in this case is not limited to opening and closing, instead it also being possible to perform more complex actions by technically more sophisticated prosthetics in the scope of the invention.

It is furthermore possible to achieve with an arm/hand prosthetic in this way all natural movements of an arm and/or hand and with the help of suitable sensors for sensing activation, spacing or temperature to reinstate both the proprioception—in other words knowledge of the location of the arm also with closed eyes—as well as the tactile and thermal sensitivity etc. of the arm.

In the second group of possible effectors 6 of special medical relevance native parts of the body are activated via functional electrosimulation as effector 6 where only the neuronal connection between the brain and the part of the body concerned is disrupted, either still intact nerve cells of the body part or directly the muscle fibers thereof being stimulated. Any feedback required can be likewise achieved either via pressure/stretch and like receptors native to the body as are still intact or by means of supportive sensors as described above for the case of activating a prosthetic. Likewise feasible also in the case of partial paralysis where, an albeit weak, remaining action capability still exists is to support these residual actions by motor-powered mechanical devices.

The third group of “virtual” effectors effector 6 is especially large, involving activation of a computer cursor or a menu selection, but also switching on a light, sending an emergency call, and the like. Feasible feedback in this case would be the cursor strike at the end of a line or page or any kind of alarm.

Particularly of interest is controlling a virtual prosthetic involving display of a bodily part three-dimensionally on a monitor and control thereof a neuronal activity of the patient or test person. By interaction with a virtual ambience the prosthetic can also be jolted or become warmer. Such events are fed back per neuronal stimulation in thus enabling in all a prosthetic to be trained and calibrated. Feedback by stimulation or by observing the effector—as applicable for calibration by means of the virtual as well as for that of a physical prosthetic—can greatly improve the activation because of learning and adaptation ability of neuronal activities (neuronal plasticity).

Once such control and simulation data is available suitable for processing by computer, thanks to Internet, of course, one is no longer limited to having to be in the vicinity, i.e. the effectors to be activated must not necessarily be in the immediate vicinity/connection to the individual controlling the effector. Thus, a prosthetic or robotic device could be displayed and controlled virtually which, in reality, is at quite a different location. Feasible are medical applications in which a surgeon can operate remotely, in military applications in which the robotic device can be controlled with high precision without danger to humans, or in contaminated or other hostile areas such as nuclear power stations, in deep sea/space technology. Although at first sight the operation and implantation in the skull for such applications would appear to be absurd, the safe and biocompatible multi-electrode 1 of the invention enhances acceptance quite considerably. And, indeed, chips are already implanted in the arm for such profane things as gaining entry to a discotheque. Thus, the time is coming when from a favorable comparison of the risks and benefits involved implanting the multi-electrode 1 will not just to be in relief from a debilitating illness.

Referring now to FIG. 7 there is illustrated a preferred embodiment of the signal interface 3. As an alternative a wired solution for data communication may be applied as is standard in neurosurgical diagnostics. But a long-term wiring solution through the surface of the body elevates the risk of infection and also from cosmetic and practical considerations is less attractive. In the preferred embodiment as described below signal communication between electrode and amplifier is by inductive energy transmission without transcutanal wiring.

The wireless signal interface 3 is divided in two, one part being above the scalp 3a, the other below. Representative of the external transceiver outside of the body, i.e. in this case above the scalp 3a only a coil 3b is shown. This external transceiver can in one embodiment simply communicate data to the amplifier 4 or the receiver 5 wireless or by a direct wired connection. Feasible is an alternative embodiment in which amplifier 4 and/or 5 are partly or completely included in a chip sited on the surface of the skull or some other suitable location on the body. Which embodiment is preferred in each case or which is at all viable will depend on the complexity of the particularly application. With current technology at least a compact transceiver connecting an external amplifier 4 or 5 over practically any distance is directly possible technically (mobile communication, Bluetooth, WLAN).

One of the communication paths can also be used for swapping data with the effector 6. Where a paralyzed natural part of the body is to be activated a further two-part signal interface similar to that as already described can be implanted in the corresponding part of the body. Since the external transceiver has facilitated access it can also be updated or replaced with more sophisticated technology without a repeat operation being needed.

Implanted below the scalp 3a as the counterpart to the external transceiver is a multi-function chip 3c as the interior transceiver. This multi-function chip 3c comprises a receiver 3c1, a transmitter 3c2 and optionally a battery 3c3. Via the lead 1b the signals from the electrodes 1c, 1d of the substrate 1a are supplied to the transmitter 3c2 and receiver 3c1 respectively.

In operation, the coil 3b of the external transceiver transmits energy and any activation signals as required for the detection electrode 1 inductively via high-frequency signals to the receiver 3c1. The multi-function chip 3c determines the control or cited stimulation signals modulated onto the communication as is known from communication technology. The energy needed for the necessary computing operations of the controllers in the multi-function chip 3c is taken from the high-frequency signals. As an alternative, the battery 3c3 or an accumulator can be inductively charged via the high-frequency signals so that the power supply is decoupled in time from the communication to the interface, requiring, of course, charging signals and stimulation signals to be kept apart in time, for instance by time windows or by separate frequency bands.

Conversely, the signals of the sensing electrodes 1c are wired via lead 1b to the transmitter 3c2 where they are relayed preferably in the signal band of 402-405 MHz of the medical implantable service band (MICS) to the coil 3b or some other item designed to receive other than coil 3b shown merely as being representative thereof.

Up to now the communication interface has been described so that the output of the transmitter 3c2 has a range only as far as the coil 3b of the external transceiver. As an alternative the transmitter 3c2 could also transmit directly to the amplifier 4 which possibly is not even sited on the surface of the skull. In this case the power supply of the multi-function chip 3c is either by long-life batteries (currently not a satisfactory solution technically) or by a charging option for instance in the way as already described by induction.

Referring now to FIG. 8 there is illustrated diagrammatically how neuronal signals are converted into effector control signals. Plotted on the left are examples of three potential profiles of three electrodes 1c. These potential signals are firstly amplified and filtered in the amplifier 4 as input signals. The filter functionality can also be localized in the system 5. As an example filter method—others are cited above in conjunction with amplifier 4—the potential signals are filtered in a native body part before being averaged over small time windows and divided up into short time windows. The activity is then analyzed by means of mathematical methods. In other words, the prediction model is determined, on the one hand, by selecting the mathematical method, on the other by calibration by means of the training data to thus obtain the intention prediction by means of the system 5. Typical mathematical methods are (1) preprocessing the signals for example a) filtering (for example low pass or bandpass), b) time/frequency analysis (e.g. Fourier transformation or multi-tapering) and/or c) binning and averaging in the time range, (2) decoding the preprocessed signals for example discriminant analysis (linear, squaring or regularized) or support vector machine (linear or radial basis function), this making no pretence to the cited methods being complete, other than the cited discriminant analysis and support vector machine being used, for example linear filter or Kalman filter particularly for decoding continual actions.

The results are the effector control signals plotted on the right, showing in this case, by way of example, two effector means, for instance two motors and the power required of them in accordance with their rotary speed.

Analysis is, of course, anything but simple. Nevertheless the person skilled in the art is aware of techniques as are already applicable, even when new and more sophisticated techniques are being developed all the time. In addition, before making use of the system 5 a training phase should be orchestrated in which the patient learns to get along with the system 1-6 and conversely to calibrate the system 5.

Referring now to FIG. 9 there is illustrated the converse data path in diagrammatically plots as examples for converting feedback data of an effector into signals for stimulating the electrodes. On the left the activation intensities of various pressure sensors and motors are plotted as a function of the time. These activation intensities are communicated as effector condition signals to the 5 where tonic pressure signals or motor activation signals are converted into phasic-tonic high-frequency stimulation signals. These stimulation signals as plotted on the right as potentials as a function of time are each communicated to one or more electrodes 1d interacting electromagnetically or stimulating the adjoining neurons also responsible for stimulation due to the targeted activation of the substrate 1a. When calibrating this system the cooperation of the patient is of help by commenting on what he feels from stimulation by various arrays of electrodes. As already explained in contact with conversion of the sensing signals, here too, sophisticating improvements is going on all the time.

In conclusion the advantages of the invention will again be summarized:

Many areals of the human cortex are not located on the surface but concealed in fissures (sulci). Electrodes shaped compatably can be implanted in such locations without displacing tissue. This can be especially relevant for a preferred embodiment in the somatosensorial cortex and motor cortex because major parts of the primary motor cortex (which play a central role in performing intentional activities and the neuronal action coding of which is best understood) are located concealed in what is called the central sulcus. In other words, proportions of the cortex located concealed in the depth of individual convolutions of the brain (including proportions of the so-called Brodmann areal amplifier 4 important for the control of intentional actions of the hand and arm) and thus attainable.

An electrode implanted here has in addition the advantage that the primary somatosensorial cortex (which receives and processes proprioceptive signals in thus contributing towards the perception of the action) is directly located opposite the motor cortex; not only that but with the same somatotopic arrangement as well (i.e. opposite the portion, for example the hand, responsible for performing the action, possibly with a displacement, the region responsible for the corresponding perception of action of the hand).

Thus, a motorized prosthetic controlled by an electrode implanted in this case intrasulcal permits additional sensorial feedback via the same electrode, now paving the way to two-way communication for a patient with minimum discomfort.

### LIST OF REFERENCE NUMERALS USED

1 multi-electrode
1a substrate
1b lead
1c (detection) electrodes
1d (stimulation) electrodes
1e conductors
2 cortex
2a somatosensorial cortex
2b primary motor cortex
3 signal interface
3a scalp
3b coil of external transceiver
3c multi-function chip
3c1 receiver
3c2 transmitter
3c3 battery/accumulator
4 amplifier
5 analyzer/central controller
6 effector
6a1 effector input lead
6a2 effector output lead
6b1 rotation system
6b2 gripper system
6c pressure sensors
6d cladding

1. A sensor for data communication between a brain and a data processor, the sensor comprising a substrate to which electrodes are applied for sensing neuronal activity and/or the transfer of stimulation in electromagnetic interaction with neurons of the brain and which can be coupled to the data processor, the substrate being shapeable to conform with an inner surface of the brain such that it can be inserted into an interior of a sulcus of the brain
wherein
the substrate is configured flexible and comprises two surfaces facing each other, on at least one of the surfaces at least one array of electrodes is applied, the electrodes being configured as contact pads such that the at least one array of electrodes can electromagnetically interact with neurons of at least one sidewall of the sulcus, the electrodes being adaptable in their array to the morphology of the at least one sidewall.

2. The sensor as set forth in claim 1 wherein both surfaces of the substrate are configured flat.

3. The sensor as set forth in claim 1 wherein applied to the two facing surfaces is a first and respectively second array of electrodes, the first array of electrodes can electromagnetically interact with neurons of the sidewall of the sulcus and the second array of with neurons of the second sidewall of the sulcus, the electrodes being adaptable in their array to the morphology of the first sidewall and second sidewall respectively.

4. The sensor as set forth in claim 1 wherein the first array comprises detection electrodes and the second array stimulation electrodes.

5. The sensor as set forth in claim 1 wherein the substrate is made of polyimide or silicone.

6. The sensor as set forth in claim 1 wherein electrodes having a density between one and 1,000 electrode contacts per cm² are applied to the substrate.

7. The sensor as set forth in claim 1 wherein the electrodes are made of gold, platinum, a metallic alloy, conductive plastics or semiconductor materials.

8. A means for data communication between a brain of a living being and a data processor comprising at least one sensor in accordance with claim 1 and a data processor configured to convert signals of the electrodes into neuron signals for processing in the data processor and/or output signals of the data processor into stimulation signals for the electrodes.

9. The means as set forth in claim 8 wherein the data processor is configured to activate a first part of the electrodes by reading out the input signals of the detection electrodes and a second part of the electrodes by means of feeding output signals as stimulation electrodes so that a two-way exchange of data is made possible.

10. The means as set forth in claim 8 wherein the data processor is additionally configured as an effector controller of a connectable effector and computes on the basis of the input signals effector control signals for the effector and/or computes on the basis of the effector control signals of the effector the stimulation signals.

11. The means as set forth in claim 10 wherein
the effector is a prosthetic;
the input signals are those of neurons in the motor cortex and the stimulation signals are for neurons in the somatosensorial cortex;
the effector control signals tweak activation parameters of the effector and
the effector condition signals are position, action and/or condition parameters of further sensors such as pressure, tactile, spacing or temperature sensors so that the brain can control activation of the prosthetic and directly receives somatosensorial feedback as to the action and ambience of the prosthetic.

12. The means as set forth in claim 10 wherein
the effector is a body part of the living being,
the input signals and those of neurons in the motor cortex and the stimulation signals are for neurons in the somatosensorial cortex;
the effector control signals tweak motor neurons or muscle fibers of the body part and
the effector condition signals are signals from receptors or receptor neurons of the body part and/or position, action and/or condition parameters of further sensors such as pressure, tactile, spacing or temperature sensors
so that the brain can control the action of the body part and directly receives somatosensorial feedback as to the action and ambience of the body part.

13. The means as set forth in claim 10 wherein
the effector is a computer particularly including a display;
the input signals and those of neurons in the motor cortex and the stimulation signals are for neurons in the somatosensorial cortex;
the effector control signals tweak virtual particularly displayed actions or functions in the computer and
the analyzer computes on the basis of the virtual action or function the effector condition signals.

14. The means as set forth in claim 8 wherein an amplifier is provided configured for amplifying and filtering input signals into preprocessed input signals and/or output signals into stimulation signals.

15. A method of producing a sensor for data communication between a brain and a data processor comprising the following steps:
mapping the geometry of a sulcus of the brain on the basis of the structural image data of the brain,
shaping the substrate to conform with the special geometry to permit insertion of the substrate into the sulcus,
mapping the neuronal activities in the region of the sulcus on the basis of functional image data of the brain;
arranging electrodes on the substrate in accordance with the mapped neuronal activities such that the electrodes can be assigned on the substrate locations in the region of the sulcus having significant neuronal activities.

16. The method as set forth in claim 15 wherein the functional image data represent the activities of neurons of the brain for a series of activation tasks, the activation tasks involving particularly observing and activation.

17. The method as set forth in claim 15 wherein the substrate is made of a flexible material.

18. The method as set forth in claim 15 wherein for a two-way exchange of data a first part of the electrodes is configured for reading out input signals as detection electrodes and a second part of the electrodes is configured for feeding output signals as stimulation electrodes.

19. The method as set forth in claim 15 wherein a first array of the electrodes is applied to the substrate for electromagnetic contact with neurons of a second sidewall and a second array of the electrodes is applied to the substrate for electromagnetic contact with neurons of a sidewall.

20. A method for data communication between a data processor and a brain of a living being wherein a sensor as set forth in claim 1 is inserted in a sulcus, the method comprising the following steps:
mapping activities and/or stimulating neurons each by electromagnetically interact with neurons of the brain;
converting the mapped signals from the electrodes into neuron signals for processing in the data processor and/or output signals of the data processor into stimulation signals for the electrodes.

21. The method as set forth in claim 20 wherein for effector control of a connected effector on the basis of the input signals the effector control signals for the effector are computed and/or on the basis of the effector condition signals of the effector the stimulation signals are computed.

22. The method as set forth in claim 21 wherein
the effector is a prosthetic;
the input signals are those of neurons in the motor cortex and the stimulation signals are for neurons in the somatosensorial cortex;
the effector control signals tweak activation parameters of the prosthetic and
the effector condition signals are position, action and/or condition parameters of further sensors such as pressure, tactile, spacing or temperature sensors, so that the brain can control activation of the prosthetic and directly receives somatosensorial feedback as to the action and ambience of the prosthetic.

23. The method as set forth in claim 21 wherein
the effector is a body part of the living being,
the input signals and those of neurons in the motor cortex and the stimulation signals are for neurons in the somatosensorial cortex;
the effector control signals tweak motor neurons or muscle fibers of the body part and
the effector condition signals are signals from receptors or receptor neurons of the body part and/or position, action and/or condition parameters of further sensors such as pressure, tactile, spacing or temperature sensors,
so that the brain can control the action of the body part and directly receives somatosensorial feedback as to the action and ambience of the body part.

24. The method as set forth in claim 21 wherein
the effector is a computer particularly including a display;
the input signals and those of neurons in the motor cortex and the stimulation signals are for neurons in the somatosensorial cortex;
the effector control signals tweak virtual particularly displayed actions or functions in the computer and
the analyzer computes on the basis of the virtual action or function the effector condition signals.

25. The method as set forth in claim 19 wherein by amplifying and filtering the input signals are converted into preprocessed input signals and/or the output signals into stimulation signals.
